(19) United States
(12) Patent Application Publication
(10) Pub. No.: US 2011/0263997 A1
(43) Pub. Date: Oct. 27, 2011

(54) SYSTEM AND METHOD FOR REMOTELY DIAGNOSING AND MANAGING TREATMENT OF RESTRICTIVE AND OBSTRUCTIVE LUNG DISEASE AND CARDIOPULMONARY DISORDERS

(75) Inventor: Stephen B. CORN, Sharon, MA (US)

(73) Assignee: ENGINEERED VIGILANCE, LLC, Sharon, MA (US)

(21) Appl. No.: 13/079,173

(22) Filed: Apr. 4, 2011

Related U.S. Application Data
(63) Continuation-in-part of application No. 11/308,675, filed on Apr. 20, 2006.
(60) Provisional application No. 61/320,561, filed on Apr. 2, 2010.

(51) Int. Cl. A61B 5/08

(52) U.S. Cl. 600/529

(57) ABSTRACT

An apparatus, system and method for non-contact monitoring of respiratory functions that is used to identify and treat restrictive and obstructive lung disease or cardiopulmonary disorders including asthma or congestive heart failure in a monitored subject from a remote location. Respiratory waveforms are generated based on non-contact monitoring of physiologic functions. The generated waveforms are analyzed and compared to waveforms to help identify the presence of cardiopulmonary disease. Inspiratory:Expiratory ratios (I:E ratios) are calculated from the generated waveform to assist remote clinicians in their diagnosis and treatment of the monitored subject.

### RELATED APPLICATION

The present application is related to and claims the benefit of U.S. Provisional Patent Application No. 61/320,561, filed Apr. 2, 2010, entitled “System and Method for Remotely Diagnosing and Managing Treatment of Restrictive and Obstructive Lung Disease and Cardiopulmonary Disorders. The present application is also a continuation-in-part of U.S. application Ser. No. 11/308,675, filed Apr. 20, 2006, and entitled “Method for Using a Non-Invasive Cardiac and Respiratory Monitoring System.” The contents of both applications are incorporated herein by reference in their entirety.

### BACKGROUND

Telemedicine technologies are useful tools for the treatment and monitoring of chronic obstructive pulmonary disease (COPD) and reactive airway diseases (RAD) such as asthma. For example, according to a study by researchers at the Veterans Administration's Medical Center (VAMC) in Milwaukee, patients believed to have COPD typically experience a 2 or 3-day process as they travel to VAMC and undergo diagnosis. In addition, some of the patients who made the long journey ultimately did not have the disease. In contrast, VAMC efforts to use a respiratory therapist or registered nurse at a remote site with patients staying closer to home have proven very successful. In ninety percent of VAMC cases, doctors reached a final diagnosis after the first consultation. Telemedicine also resulted in significant changes in medication, diagnostic procedures or lifestyle for many of the patients in the study. Notably, the use of telemedicine saved 684 patient visits during the seven year study period and more than 294,000 miles of travel and 748 work days.

### BRIEF SUMMARY OF THE INVENTION

The embodiments of the present invention provide a mechanism for remote non-contact monitoring of physiologic (e.g.: respiratory and cardiac) functions that may be used to diagnose and monitor restrictive and obstructive lung disease including pneumonia, COPD and reactive airway diseases such as asthma and croup, and cardiopulmonary disorders that impact on the respiratory system, such as congestive heart disease. Respiratory waveforms are generated based on the monitored physiologic functions. Inspiratory to expiratory (I:E) ratios which are an important tool in determining the presence or absence of restrictive and obstructive lung disease are determined from the waveform and displayed. Analysis can be performed at the monitoring site or the gathered waveform may be transmitted to a location remote from the monitoring site for analysis. The generated waveforms and ratios may be analyzed and compared to a target waveform for the disease to help identify whether the subject has restrictive or obstructive lung disease and/or determine the current status of the individual.

Comparison to a patient's own waveform over time may also form part of the analysis and treatment process. For example, a patient may be monitored in the morning to establish a baseline waveform, use a bronchodilator or undergo other treatment, and then be monitored to capture a second waveform. The response to therapy for that individual (comparing an earlier to later waveform) will help a clinician determine if therapy should be altered or maintained or if the patient needs to come in and be re-evaluated in-person by a clinician.

In one embodiment, a monitoring and analysis system for identifying and treating restrictive and obstructive lung disease or cardiopulmonary disorders from a remote location includes a respiratory waveform detection module. The respiratory waveform detection module performs non-contact monitoring of a subject to detect respiratory motion and generates a waveform based on the detected respiratory motion. The system also includes an analysis module programmatically analyzing the generated waveform to identify restrictive and obstructive lung disease or cardiopulmonary disorders in the monitored subject. The analyzing process also calculates an inspiratory:expiratory (I:E) ratio from the generated waveform and compares the generated waveform to a stored waveform indicative of restrictive and obstructive lung disease or cardiopulmonary disorders. A result of the comparison and the I:E ratio is displayed to a remotely located clinician for further analysis.

In another embodiment, a computing-device implemented method for identifying and treating restrictive and obstructive lung disease or cardiopulmonary disorders from a remote location includes the performing of non-contact monitoring of a subject to detect respiratory motion. The method also generates programmatically a waveform based on the detected respiratory motion and calculates programmatically an inspiratory:expiratory (I:E) ratio from the generated waveform. The generated waveform is programmatically compared to a stored waveform indicative of restrictive and obstructive lung disease or cardiopulmonary disorders to identify whether the monitored subject is afflicted with restrictive and obstructive lung disease or cardiopulmonary disorders. The method also displays the calculated I:E ratio and a result of the comparing to a remotely located clinician for further analysis.

In an embodiment, a method for identifying and treating lung or cardiopulmonary disorders from a remote location includes performing non-contact monitoring of a subject to detect respiratory motion of a subject. The method also generates programmatically a waveform based on the detected respiratory motion and analyzes programmatically the generated waveform to identify or monitor lung or cardiopulmonary disorders. Based on the analyzing, an order is programmatically transmitted to dispense medication to the subject.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1 depicts an exemplary environment suitable for practicing embodiments of the present invention;

FIG. 2 depicts an exemplary integrated monitoring apparatus;

FIG. 3 depicts an exemplary sequence of steps performed by an embodiment of the present invention to identify and/or monitor individuals with restrictive and obstructive lung disease or cardiopulmonary disorders;

FIG. 4 depicts user-customizable music and image settings on a smartphone; and

FIGS. 5A-5C depict exemplary aroma dispensing modules.

### DETAILED DESCRIPTION

The embodiments of the present invention utilize a non-contact monitoring system to remotely monitor physiologic functions of a monitored subject. The functions are analyzed to diagnose the presence and/or status of restrictive and obstructive lung disease or cardiopulmonary disorders in a monitored subject. Non-contact measurement of breathing parameters (e.g.: rate, rhythm amplitude, pauses, inspiratory to expiratory ratio, breathing frequency variability) and/or body movements are used in the diagnostic process. Feedback can be provided to the monitored subject in real-time from doctors in remote locations and treatments and therapies may be adjusted as needed.

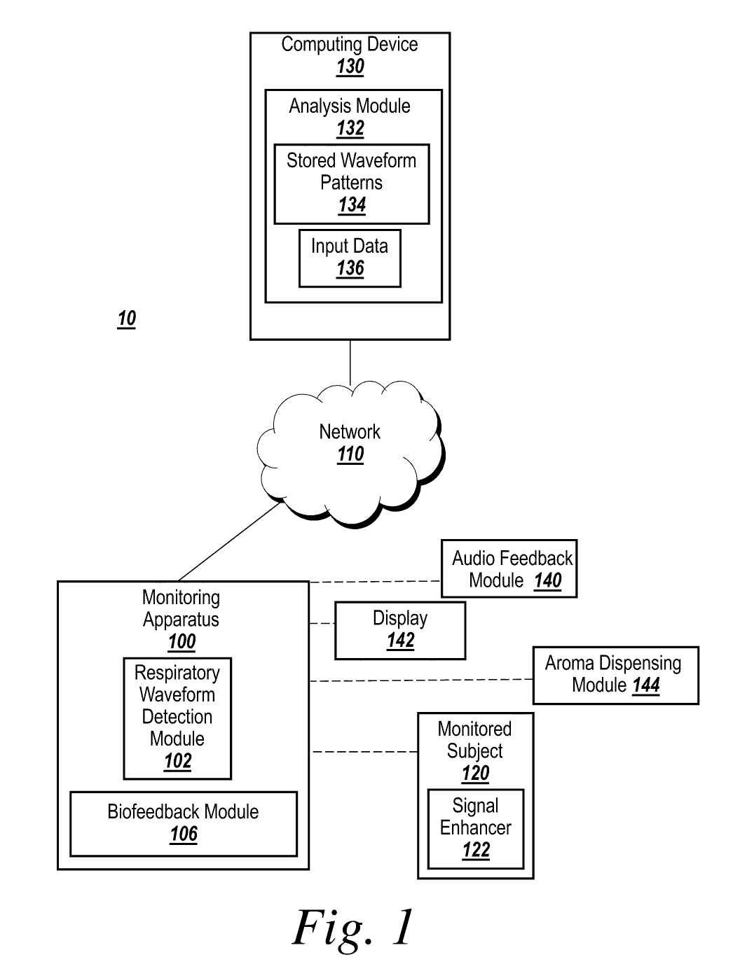

FIG. 1 depicts an exemplary environment suitable for practicing embodiments of the present invention. Monitoring system 10 may include monitoring apparatus 100 that is used to monitor physiological factors for a monitored subject 120. Monitoring apparatus 100 may include a respiratory waveform detection module 102. Respiratory waveform detection module 102 is used to perform non-contact respiratory monitoring of monitored subject 102 and to generate a waveform representing the monitored respiratory process. A number of different techniques to perform the non-contact monitoring may be used and are described in greater detail below.

In order to get the subject into a relaxed state for monitoring, the monitoring system may include biofeedback module 106. Biofeedback module 106 may provide alternative sensory feedback designed to create an environment conducive to achieving or maintaining a desired respiratory status (for example to calm down a subject during an asthmatic event or prevent the onset of the event or to allow for easier monitoring). For example, biofeedback module 106 may provide visible displays, audible feedback such as music via audio module 140, or aromatic feedback via aroma dispensing module 144, that is designed to assist the monitored subject in achieving a desired breathing status. In one embodiment, the biofeedback may occur in the form of a voice giving algorithm-based guidance to a subject to attempt to lead the subject in a breathing exercise in order to bring the subject's breathing closer to a desired respiration rate and thereby achieve a target waveform. By utilizing voice-based instruction, the subject may perform the breathing exercise with his or her eyes closed and avoid visual distractions that might otherwise be present.

Once a waveform representing the monitored respiratory function has been generated, monitoring system 10 analyzes the generated waveform to determine whether the current monitored physiologic process is indicative of restrictive and obstructive lung disease or cardiopulmonary disorders. In one embodiment, the generated waveform is programmatically analyzed by a software analysis module 132 executing on a computing device 130. Computing device 130 may take many forms, including but not limited to a personal computer, workstation, server, network computer, quantum computer, optical computer, bio computer, Internet appliance, mobile phones and other mobile devices such as smartphones, a pager, a tablet computing device, or other form of computing device equipped with a processor and able to execute analysis module 132. Computing device 130 may be electronic and may include a Central Processing Unit (CPU), memory, storage, input control, modem, network interface, etc. The CPU may control each component of computing device 130 to provide an environment suitable for executing analysis module 132. The memory on computing device 130 temporarily stores instructions and data and provides them to the CPU so that the CPU operates the computing device 130.

Optionally, computing device 130 may include multiple CPUs for executing software loaded in memory and other programs for controlling system hardware. Each of the CPUs can be a single or a multiple core processor. The code loaded in the memory may run in a virtualized environment, such as in a Virtual Machine (VM). Multiple VMs may be resident on a single processor. Also, part of the code may be run in hardware, for example, by configuring a field programmable gate array (FPGA), using an application specific instruction set processor (ASIP) or creating an application specific integrated circuit (ASIC).

Input control for the computing device 130 may interface with a keyboard, mouse, microphone, camera, such as a web camera, or other input devices such as a 3D mouse, space mouse, multipoint touchpad, accelerometer-based device, gyroscope-based device, etc. Computing device 130 may receive, through the input control, input data 136 relevant for calculating target waveforms for monitored subject 120. Optionally, computing device 130 may display data relevant to the generated waveform on a display as part of the analysis process.

In one embodiment, monitoring apparatus 100 communicates with computing device 130 over a network 110. Network 110 may be the Internet, an intranet, LAN (Local Area Network), WAN (Wide Area Network), MAN (Metropolitan Area Network), wireless network or some other type of network over which monitoring apparatus 100 and computing device 130 can communicate. Although depicted as a separate device in FIG. 1, it should also be appreciated that computing device 130 may also be part of an integrated apparatus with monitoring apparatus 100.

Analysis module 132 analyzes the generated waveform produced by monitoring apparatus 100. The generated waveform is compared against stored waveform patterns 134 to determine whether the current generated waveform is indicative of restrictive and obstructive lung disease or cardiopulmonary disorders. The selection of the comparison waveform from the stored waveform patterns may utilize previous input data 136 that includes information regarding the monitored subject such as a previously stored base-line breathing waveform, personal medical information (e.g. sex, height, weight, age, family history of various diseases, etc. and occupational information). The information may be subject-specific or based on the subject's personal or occupational demographic. Based on available data, the analysis module 132 selects either a previously stored base-line breathing waveform, a customized target waveform or a default waveform for comparison to the generated waveform. For example, in one embodiment, the subject may be monitored to establish a baseline waveform prior to undergoing a particular treatment modality. Subsequent waveforms may then be compared to the baseline waveform to determine treatment effectiveness. In another embodiment, the stored waveforms are those of other individuals previously diagnosed as having a restrictive and obstructive lung disease or cardiopulmonary disorder.

In one embodiment, the analysis of the generated waveform may be a programmatic process that occurs in an automated fashion. In an alternate embodiment, the process may also involve human input in reviewing the selection of the target waveform and interpreting the result of the comparison prior to completion of the analysis. In one embodiment, all of the analysis decisions are saved for future study in order to continually refine the stored waveform patterns 134. It should be noted that the analysis module 132 may be located on the local monitoring device or “off-site” at a remote location.

The results of the analysis performed by the analysis module 132 may be provided to one or more remotely located clinicians. In addition to displaying the captured breathing waveform, the analysis module 132 may also calculate an inspiratory/expiratory (I:E) ratio from the captured waveform and display it to the clinician and/or overlay the display of the captured waveform with the comparison waveform for review. It should be appreciated that in some embodiments, the functionality attributed to the analysis module 132 and the respiratory waveform detection module 102 may be split into additional modules or combined into one module without departing from the scope of the present invention.

Following the analysis of the generated waveform, the clinician may take a number of actions. The clinician may do nothing and continue to monitor the subject. Alternatively, the output of the analysis module may help the clinician reach an initial diagnosis as to whether or not the monitored subject has restrictive and obstructive lung disease or cardiopulmonary disorders. Additionally, the output of the analysis module may indicate to the clinician that a monitored subject who has previously been diagnosed with restrictive and obstructive lung disease or cardiopulmonary disorders has undergone a change in condition with the result that the clinician prescribes different treatment and/or medicines for the monitored subject. The embodiments of the present invention thus allow real-time monitoring and treatment of individuals with restrictive and obstructive lung disease or cardiopulmonary disorders from a remote location.

In one embodiment, the non-contact monitoring system may also be configured to measure “phase difference”. Phase difference measures changes in the abdomen and thorax that occur during breathing that are a sign of breathing disorders including asthma. The synchrony or asynchrony between chest and abdomen during breathing has wide diagnostic and therapeutic implications for respiratory disease, sleep disturbances, recovery from anesthesia and may provide predictive and biofeedback information for relaxation and stress reduction techniques. Such measurements may be used to monitor response to treatment from a remote location. The measured data would be objective (as opposed to the clinician simply observing as in the present standard-of-care) and could be stored and made a part of the monitored subject's chart for future reference.

The non-contact monitoring system may use radiated energy (e.g.: ultrasonic, radio frequency, infrared, laser, etc.) to identify respiratory waveforms in patients. The monitoring system may illuminate a subject in radiated energy and then detect the reflected radiated energy caused by respiratory functions. Of note, the breathing waveform can be captured through clothes and does not need a specific window to receive the necessary information to generate a breathing waveform. However, in one embodiment, a signal enhancer 122 may be utilized to augment the reflected signal. This may be in the form of a “relaxation patch” worn by the participant. The detected reflections are used to plot a two-dimensional waveform. The waveforms represent the rise and fall of a detected signal (the reflected energy) over time and are indicative of the small movements of the patient's chest, abdomen and/or other anatomical sites that are associated with respiratory function. Different implementations of the monitoring system use different forms of radiated energy (e.g.: laser, ultrasonic energy and radio frequency) to capture breathing waveforms for analysis.

One example of a suitable non-contact monitoring system that may be leveraged in conjunction with the embodiments of the present invention is described in U.S. Pat. No. 6,062,216 ('216 patent). As described in the '216 patent, a respiratory monitor may employ either ultrasonic or laser monitoring of an individual's breathing function by measuring changes in body position with respect to time. The device continuously and without the need for contact, monitors the individual's breathing function (and analyzes the measured waveform and identifies respiratory rate, apneic pauses, and obstructive breathing) and body movements. The '216 patent (the contents of which are hereby incorporated by reference) describes a monitoring system using laser energy or ultrasonic energy to monitor respiratory function so as to detect sleep apnea but may be adapted to perform the respiratory monitoring described herein. It should be appreciated that although the monitoring system of the '216 patent has been cited as an exemplary monitoring system which may be used in the present invention, other non-invasive monitoring systems utilizing laser or ultrasonic energy to detect respiratory waveforms may also be used within the scope of the present invention.

In one embodiment, the respiratory waveform detection module 102 may use ultrasound to perform the physiological monitoring to establish the waveforms used in the present invention. Ultrasonic sound is a vibration at a frequency above the range of human hearing, in other words usually in a range above 20 kHz. In one embodiment, a shaped transducer in the monitoring system radiates a preferably continuous beam of ultrasound for example in the 25 kHz to 500 kHz range to illuminate a subject patient. A receiving transducer in the monitoring system of the present invention or transducer array develops one or more signals, which shift slightly from the incident frequency due to respiratory motion. The signal is then analyzed and plotted to generate a waveform, which may be compared against an appropriate benchmark. Appropriate adjustments are made by the monitoring system to account for the distance between the monitoring system and the subject as well as any environmental factors affecting the detection of the reflected energy.

In another embodiment, the monitoring system may use laser detection means as described in the '216 patent in place of ultrasonic energy. In such a case a laser illuminates the subject patient in a beam of light of a selected wavelength and the reflected energy, which varies based on respiratory movements is traced so as to generate a waveform. Additionally, other embodiments utilizing infrared, radio frequency or other wavelength ranges in the electromagnetic spectrum may be employed in order to perform the non-contact monitoring and analysis of respiratory functions described herein.

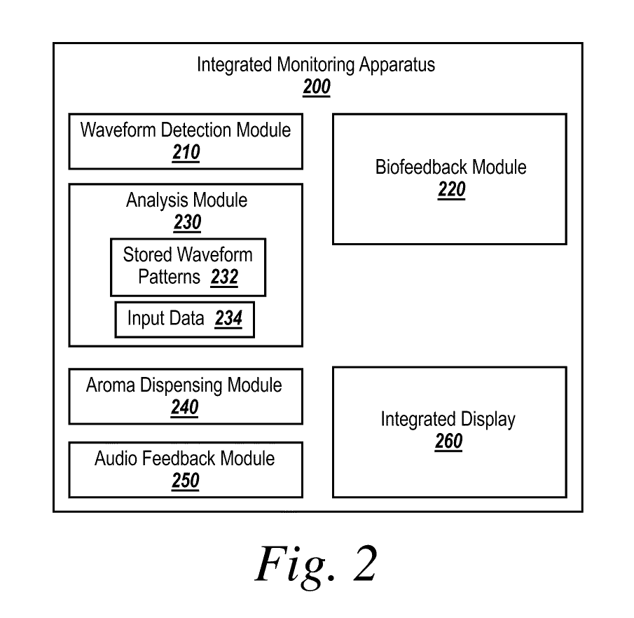

In one embodiment, the monitoring system described herein may be provided as an integrated monitoring apparatus rather than as separate components in multiple devices. FIG. 2 depicts an exemplary integrated monitoring apparatus 200 that includes most or all of the components of the monitoring system described in FIG. 1. The integrated monitoring apparatus 200 may include one or more waveform detection modules 210 such as respiratory waveform detection modules. The integrated monitoring apparatus 200 may also include biofeedback module 220 and analysis module 230. Analysis module 230 may include stored waveform patterns 232 and stored input data 234 specific to a monitored subject. It will be appreciated that the waveform detection module 210, biofeedback module 220 and analysis module 230 may be combined into a single module or split into additional modules without departing from the scope of the present invention.

In one embodiment, integrated monitoring apparatus 200 may also include an aroma dispensing module 240 and an audio module 250 for providing aromatic and audio feedback and an integrated display module 260 utilized to provide visual feedback to a monitored subject in the manner described herein. In other embodiments, integrated monitoring apparatus 200 may contain some but not all of the modules 240, 250 and 260 used to provide feedback and biofeedback. The aroma dispensing module 240 may include one or more stored scents that are designed to be soothing when inhaled and that are released into the monitored subject's environment at different times and in different amounts upon a signal being received from the biofeedback module 206. In an additional aspect of an embodiment of the present invention, the tactile, audible, visual and aromatic feedback may be dispensed as an adjunct to monitoring to prepare the subject for monitoring by creating a proper mood for monitoring prior to, or in addition to, any monitoring-based biofeedback being delivered.

In one exemplary embodiment, the integrated monitoring apparatus 200 may be provided via a portable device such as a mobile phone or smartphone, tablet computing device or laptop. For example, the mobile phone or smartphone, tablet computing device or laptop may be equipped with an ultrasound probe that is part of the device or connected via BLUETOOTH, or connected via a USB or other interface and that that is used to perform ultrasound monitoring. The detection and/or analysis modules described herein may be pre-installed or downloaded to the device. In one embodiment, portable devices such as a mobile phone or smartphone, tablet computing device or laptop display and speakers may be used to provide visual, audio and/or tactile feedback.

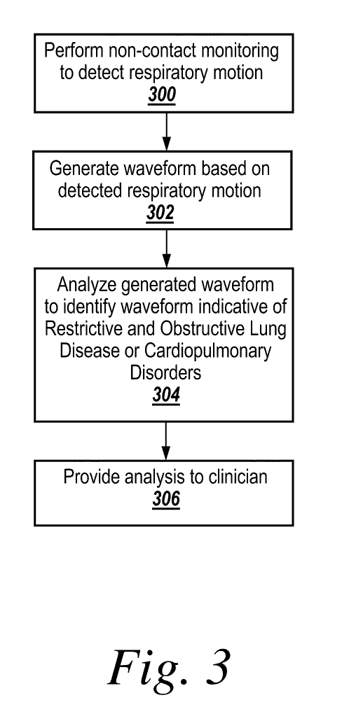

FIG. 3 depicts an exemplary sequence of steps performed by an embodiment of the present invention to identify or monitor restrictive and obstructive lung disease or cardiopulmonary disorders in a monitored subject. The sequence may begin by providing non-contact monitoring of a subject as described herein to detect respiratory motion (step 300). Of note, the subject may or may not be aware of the monitoring. In one embodiment, the subject is informed of the beginning of the monitoring. The subject in such a case may attempt to perform breathing exercises to enter a relaxed state. In another embodiment, background monitoring may be conducted as part of a normal background process. For example, the monitoring could be performed continually at work.

After data is gathered, a waveform is generated as a result of the monitoring process (step 302). The waveform is analyzed by the analysis module to identify whether the subject being monitored has restrictive and obstructive lung disease or cardiopulmonary disorders or whether their condition has changed (step 304). The analysis may include the generation of an I:E ratio from the generated waveform. The analysis is provided to a clinician for further examination (step 306). Optionally, the clinician can then contact the subject to inform the subject in real-time of the results. In one embodiment, a patient may provide a sample breathing waveform, then receive treatment such as using a bronchodilator, and then send another waveform for analysis. The analysis may compare the later waveform to the earlier waveform to determine if therapy should be increased, stay the same, be decreased, changed or if a patient needs to be seen in person.

As noted above, the embodiments of the present invention may be used to determine I:E ratios for a monitored subject. In one embodiment, the I:E ratio is calculated as the quotient of the duration of time that the target surface is moving away from the sensor (assumed to be expiration) and the duration of time the target is moving towards the sensor (assumed to be inspiration). Inspiration and expiration durations are monitored over several full breathing cycles so that the resulting measurement is an average. The ratio is displayed on the screen whenever a sufficient number of full breathing waveforms that pass a simple quality screen have been counted. The ratio is displayed as “1:X”—where X is the calculated quotient, in one embodiment rounded to the nearest 0.25.

For example, in one embodiment designed to run on an IPHONE acting as a monitoring device as described herein, the sample-by-sample processing of the filtered ultrasound signal (out) and the calculated output suppression variable (sup) that are currently being used in the IPHONE code (which may be written in MATLAB, a programming language and environment provided by The MathWorks, Inc. of Natick, Mass.) may be expressed as:

For each sample of the displayed breathing waveform, utilizing the current and previous value of the out signal and the current value of the sup variable, do the following:

| | |
|---|---|
| - | if sup flag is on (output to be suppressed) then |
| - | clear all counters and variables |
| - | goto next sample |
| - | else |
| - | increment curdur. |
| - | compare current and prior out samples to determine if current sample is |

inspiratory (>= prior out) or expiratory (< prior out).

| | |
|---|---|
| - | if current sample is inspiratory, then |

| | |
|---|---|
| - | increment curindur. |
| - | if ( lastie was expiratory ) AND ( out <= −transthresh ) , then |

| | |
|---|---|
| - | if ( durthresh1 <= curdur <= durthresh2 ), then |

| | |
|---|---|
| - | increment goodcyc. |
| - | copy curindur and curexdur to head position of indurvec |

and exdurvec respectively.

| | |
|---|---|
| - | clear curdur, curindur, and curexdur. |
| - | if goodcyc >= numcyc, then |

| | |
|---|---|
| - | calculate raw I:E as ierat = |

sum(exdurvec)/sum(indurvec).

| | |
|---|---|
| - | round ierat to nearest 0.25. |

| | |
|---|---|
| - | else |

| | |
|---|---|
| - | clear all counters and variables |
| - | goto next sample |

| | |
|---|---|
| - | set lastie to inspiratory (+1). |

| | |
|---|---|
| - | else |

| | |
|---|---|
| - | increment curexdur. |
| - | set lastie to expiratory (−1). |

where:
numcyc - number of quality full breath cycles required to enable calculation

and display of ratio.

transthresh - threshold amplitude required to classify a point as a

transition from insp. to exp.

durthresh1 - minimum duration of full breath cycle to include in I:E ratio

calculation.

durthresh2 - maximum duration of full breath cycle to include in I:E ratio

calculation.

lastie - flag indicating whether the last sample point was insp.(+1), exp.(−

1), or undef (0).

curdur - duration of current breath cycle.
curindur - count of the number of samples classified as inspiration in

current breath.

curexdur - count of the number of samples classified as expiration in

current breath.

indurvec - length numcyc circular buffer of inspiration durations.
exdurvec - length numcyc circular buffer of inspiration durations.
goodcyc - counter of quality full breathing cycles.
ierat - calculated I:E ratio. Value of zero indicates that ratio should not be

displayed.

It should be noted that although much of the discussion herein has focused on lung disorders, other disorders, such as congestive heart disease (cardiopulmonary), which will affect breathing (impacting respiratory rate, amplitude, I:E ratio, and breathing rate variability) and other diseases that indirectly impact respiration may be monitored using the embodiments of the present invention.

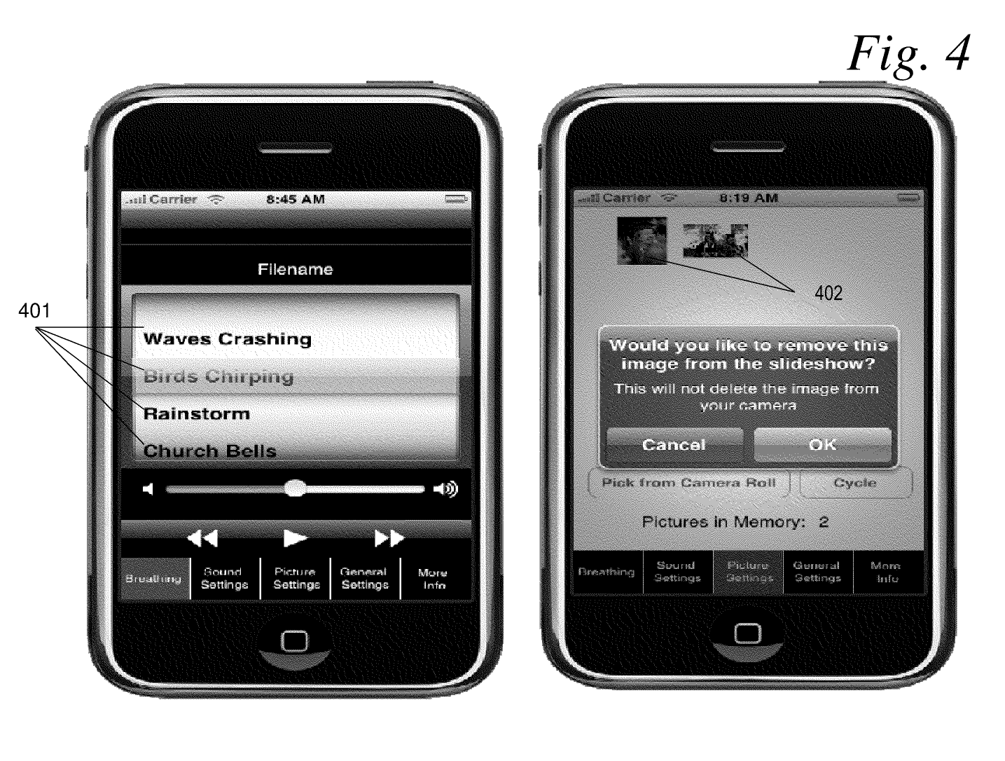

As noted above, embodiments of the present invention include components to assist in relaxing a subject for monitoring. Accordingly, in another aspect of the present invention, the system is also well suited to apply known therapies for relaxation and stress reduction. That is, getting the participant to reduce his or her breathing rate can be coupled with tactile, visual, aromatic or auditory cues to relax the subject. Visual, aromatic, audible and tactile feedback may be delivered to the subject with or without a waveform being displayed. Music or visual images can be displayed in response to both positive and negative physiologic responses on the system. In one embodiment, the form of the music, visual images or other biofeedback being presented to a user may be customizable by the user. For example, in one embodiment, the user may adjust settings on a mobile phone being utilized as the integrated monitoring apparatus of the present invention. For example, as depicted in FIG. 4, the user may select the particular audio selections 401 or images 402 presented in a slideshow that are generated by the phone in response to monitored physiological responses.

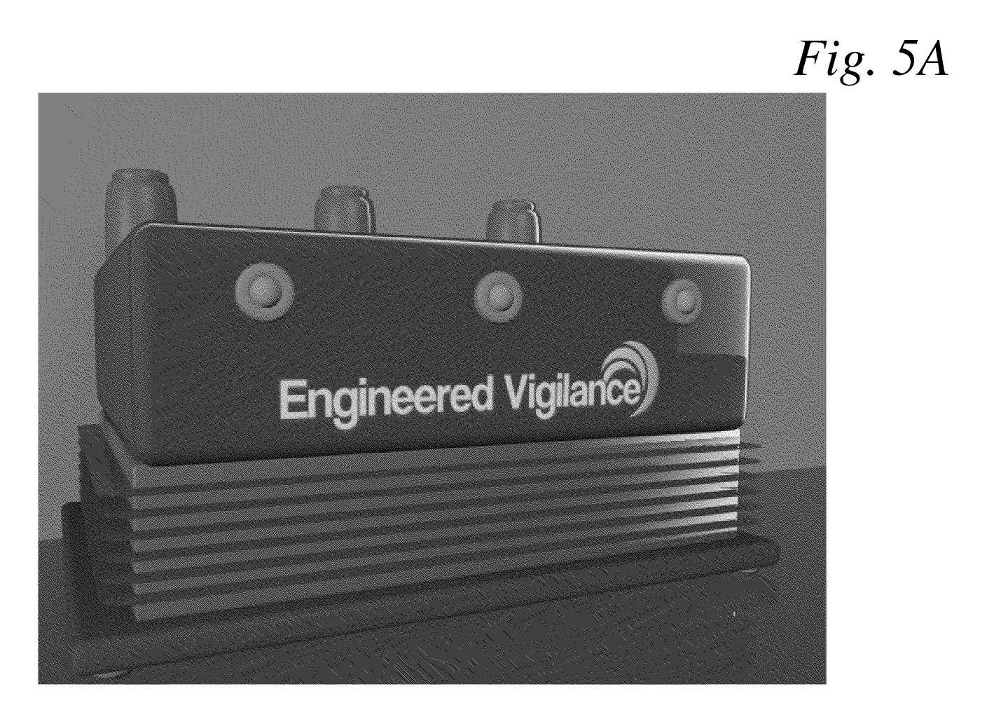

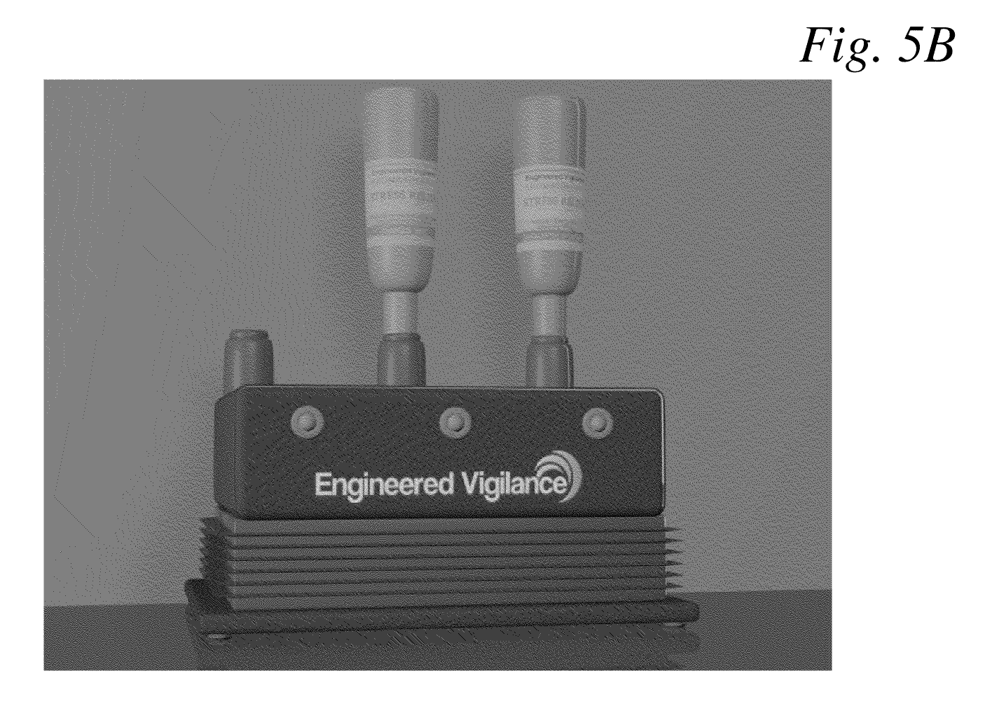

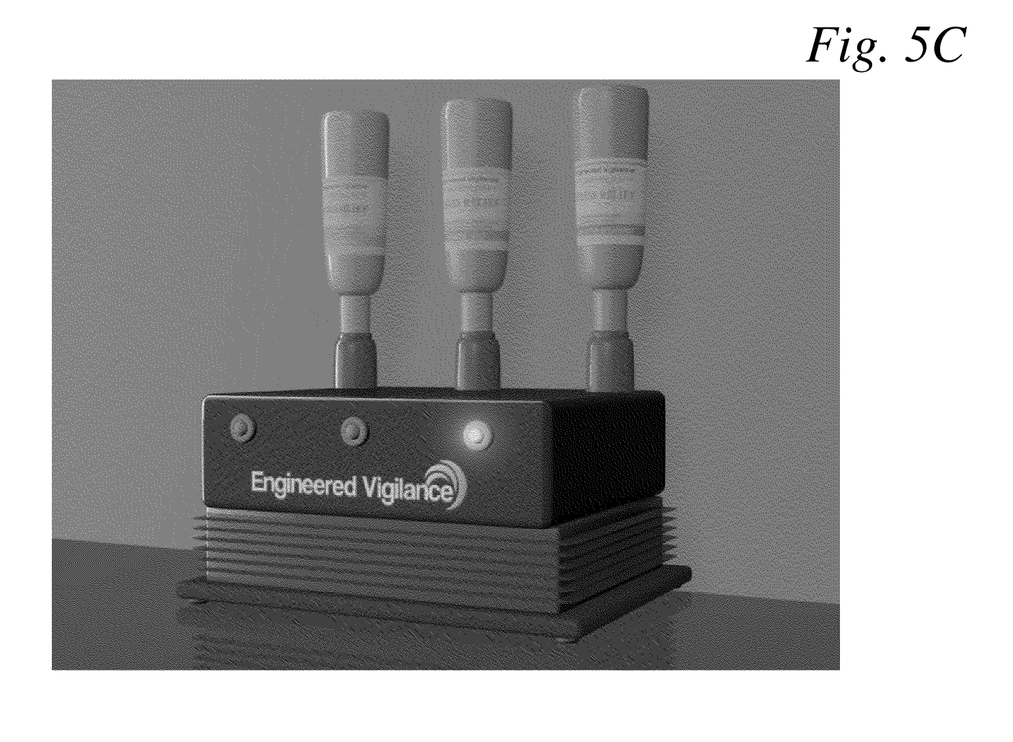

In another embodiment, the tactile, visual, aromatic or auditory cues may be delivered with or without actively monitoring the subject and/or making the subject aware of their own breathing. For example, the tactile, visual, aromatic or auditory cues may be delivered to help with stress reduction or relaxation for an individual. For instance, aromatherapy is a form of alternative medicine that uses aromatic compounds for the purpose of improving a person's mood, cognitive function and health. Aromatherapy has been shown to be instrumental in enhancing stress management and relaxation programs. In one embodiment an aromatherapy module may be integrated with personalized stress management programs. The module holds one or more generic or propriety essences. The release of the relaxing essences may be initiated by apps which can be running on desktop computers, laptops, Smartphones or tablet computing devices such as the IPAD that are in communication with, or integrated with, an aroma dispensing module. FIGS. 5A-5C depict exemplary aroma dispensing modules. It will be appreciated that the aroma dispensing module may also be functioning as the monitoring device as described herein.

In one programmed mode, soothing and refreshing essences are released into the user's environment a short period of time before the user is notified that it is time to enter a relaxation exercise or be monitored. In this manner, the user “gets-in-the-mood” and is psychologically prepared and primed to perform the relaxation exercise. Release of the aromatherapy can be based on monitored biofeedback from a monitoring device performing respiratory monitoring or simply based on personalized programming from the user. For example, individuals in a corporate setting may prefer to have set time periods for release of the aromatherapy, such an hour or two into the work day, early afternoon and then again before the evening commute. Alternatively, factory set time default periods may also be employed. From a consumer point-of-view, personalizing and automating the experience lead to increased compliance and well-being with relaxation, stress management, exercise and training regimens. From a corporate point-of-view, the user gets the benefits of the proprietary aromatherapy while ensuring regular and sustained use of the products.

As noted above, the monitored information and analysis decisions may be stored. The ability to store the monitored information allows an objective response to therapy, provides storage for medical records and is of importance for third party reimbursement. Further, having objective and permanent records of responses to therapy adds to the attractiveness of the technique to clinicians and leads to better compliance with a therapeutic or relaxation regime from subjects being monitored.

It should be understood that other physiologic parameters, (video, audio, etc) could be incorporated to add robustness to the proposed system. Further, though breathing and body movement are optimally derived through non-contact means to prevent the creation of an overly artificial environment, a contact monitoring system may also be used to perform monitoring of a subject.

In one embodiment, the non-contact monitoring system of the present invention may be used to diagnose and treat restrictive and obstructive lung disease and cardiopulmonary disorders in pediatric patients. For example, the monitoring system may be employed on a tablet computing device or smartphone on which an instructional cartoon or educational game is displayed in order to capture a child's attention and increase the likelihood of compliance and successful completion. During the display of the cartoon or educational game the child's breathing waveform may be captured, analyzed and transmitted (or transmitted and then analyzed depending on implementation of the monitoring and analysis system described herein).

In some embodiments, the monitoring and/or analysis modules may be deployed to the monitoring device as downloadable applets. For example, in one implementation, the monitoring and/or analysis modules may be downloaded to a smartphone or tablet computing device from a third party vendor, such as via the Apple iTunes® website.

In one embodiment, after the process of capturing the waveform and analyzing the waveform, medication is dispensed to the patient (dose, type, frequency) based on either a clinician reading the waveform or an automatic computer reading of the waveform. The dispensing of medication may occur manually or may occur through the transmission of an electronic signal to a dispensing apparatus proximally located near the monitored subject. For example, a signal might be sent to an IV unit equipped with medication connected to the monitored subject.

The present invention may be provided as one or more computer-readable programs embodied on or in one or more non-transitory physical mediums. The mediums may be a floppy disk, a hard disk, a compact disc, a digital versatile disc, a flash memory card, a PROM, an MRAM, a RAM, a ROM, or a magnetic tape. In general, the computer-readable programs may be implemented in any programming language. Some examples of languages that can be used include C, C++, C#, Python, FLASH, JavaScript, or Java. The software programs may be stored on, or in, one or more mediums as object code. Hardware acceleration may be used and all or a portion of the code may run on a FPGA, an Application Specific Integrated Processor (ASIP), or an Application Specific Integrated Circuit (ASIC). The code may run in a virtualized environment such as in a virtual machine. Multiple virtual machines running the code may be resident on a single processor.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

In addition, implementations consistent with principles of the invention can be implemented using devices and configurations other than those illustrated in the figures and described in the specification without departing from the spirit of the invention. Devices and/or components may be added and/or removed from the specifically disclosed implementations depending on specific deployments and/or applications.

We claim:

1. A monitoring and analysis system for identifying and treating restrictive and obstructive lung disease or cardiopulmonary disorders from a remote location, comprising:
a respiratory waveform detection module, the respiratory waveform detection module performing non-contact monitoring of a subject to detect respiratory motion and generating a waveform based on the detected respiratory motion;
an analysis module programmatically analyzing the generated waveform to identify restrictive and obstructive lung disease or cardiopulmonary disorders in the monitored subject, the analyzing calculating an inspiratory:expiratory (I:E) ratio from the generated waveform and comparing the generated waveform to a stored waveform indicative of restrictive and obstructive lung disease or cardiopulmonary disorders, a result of the comparison and the I:E ratio displayed to a remotely located clinician.

2. The system of claim 1 wherein the respiratory waveform detection module and the analysis module communicate over a network.

3. The system of claim 1 wherein the analysis further compares the generated waveform to a stored waveform of the subject captured during a previous monitoring period.

4. The system of claim 1 in which the I:E ratio is calculated as a quotient of a duration of time that a target surface is moving away from a sensor and a duration of time the target is moving towards the sensor.

5. The system of claim 1 wherein the respiratory waveform detection module monitors the subject using radiated energy.

6. The system of claim 5 wherein the respiratory waveform detection module monitors the subject using ultrasound.

7. The system of claim 5 wherein the respiratory waveform detection module monitors the subject using one of laser detection, infrared or radio frequency transmissions.

8. A computing-device implemented method for identifying and treating restrictive and obstructive lung disease or cardiopulmonary disorders from a remote location;
performing non-contact monitoring of a subject to detect respiratory motion;
generating programmatically a waveform based on the detected respiratory motion;
calculating programmatically an inspiratory:expiratory (I:E) ratio from the generated waveform;
comparing programmatically the generated waveform to a stored waveform indicative of restrictive and obstructive lung disease or cardiopulmonary disorders to identify whether the monitored subject is afflicted with restrictive and obstructive lung disease or cardiopulmonary disorders, and
displaying the calculated I:E ratio and a result of the comparing to a remotely located clinician.

9. The method of claim 8 wherein the comparing uses a previous breathing waveform, or personalized data concerning an occupation or physical condition of the subject.

10. The method of claim 8 in which the I:E ratio is calculated as a quotient of a duration of time that a target surface is moving away from a sensor and a duration of time the target is moving towards the sensor.

11. The method of claim 8 wherein the non-contact monitoring monitors the subject using radiated energy.

12. The method of claim 11 wherein the non-contact monitoring monitors the subject using one of ultrasound, laser detection, infrared or radio frequency transmissions.

13. The method of claim 8 wherein the non-contact monitoring detects a phase difference by measuring a change in the position of the abdomen and thorax.

14. A non-transitory computer-readable medium holding computer-executable instructions for identifying and treating restrictive and obstructive lung disease or cardiopulmonary disorders from a remote location, the instructions when executed causing at least one computing device to:
perform non-contact monitoring of a subject to detect respiratory motion;
generate programmatically a waveform based on the detected respiratory motion;
calculate programmatically an inspiratory:expiratory (I:E) ratio from the generated waveform;
compare programmatically the generated waveform to a stored waveform indicative of restrictive and obstructive lung disease or cardiopulmonary disorders to identify whether the monitored subject is afflicted with restrictive and obstructive lung disease or cardiopulmonary disorders, and
display the calculated I:E ratio and a result of the comparing to a remotely located clinician.

15. The medium of claim 14 wherein the comparing uses a previous breathing waveform captured during a previous monitoring of the subject, or personalized data concerning an occupation or physical condition of the subject.

16. The medium of claim 14 in which the I:E ratio is calculated as a quotient of a duration of time that a target surface is moving away from a sensor and a duration of time the target is moving towards the sensor.

17. The medium of claim 14 wherein the non-contact monitoring monitors the subject using radiated energy.

18. The medium of claim 17 wherein the non-contact monitoring monitors the subject using one of ultrasound, laser detection, infrared or radio frequency transmissions.

19. The medium of claim 14 wherein the non-contact monitoring detects a phase difference by measuring a change in the position of the abdomen and thorax.

20. A method for identifying and treating lung or cardiopulmonary disorders from a remote location, comprising:
performing non-contact monitoring of a subject to detect respiratory motion of the subject;
generating programmatically a waveform based on the detected respiratory motion;
analyzing programmatically the generated waveform to identify or monitor lung or cardiopulmonary disorders; and
programmatically transmitting an order to dispense medication to the subject based on the analyzing.
